(19) United States
(12) Patent Application Publication
(10) Pub. No.: US 2026/0355282 A1
(43) Pub. Date: Nov. 12, 2026

(54) HETERODIMERIC FUSION PROTEIN AND USE THEREOF

(71) Applicant: SHENGHE (CHINA) BIOPHARMACEUTICAL CO., LTD., Nanjing, Jiangsu (CN)

(72) Inventors: Chongbing WU, Nanjing (CN); Liusong YIN, Nanjing (CN); Xiaoling JIANG, Nanjing (CN)

(73) Assignee: SHENGHE (CHINA) BIOPHARMACEUTICAL CO., LTD., Nanjing, Jiangsu (CN)

(21) Appl. No.: 19/118,436

(22) PCT Filed: Oct. 10, 2023
(86) PCT No.: PCT/CN2023/123701

§ 371 Date: Apr. 4, 2025

(30) Foreign Application Priority Data
Oct. 10, 2022 (WO) PCT/CN2022/124255

(51) Int. Cl. A61K 38/20; A61K 38/00; A61P 35/00; C07K 14/54; C07K 16/28; C07K 16/30; C07K 16/32

(52) U.S. Cl. CPC A61K 38/2066; A61P 35/00; C07K 14/5428; C07K 16/28; C07K 16/2803; C07K 16/2863; C07K 16/2875; C07K 16/2878; C07K 16/2896; C07K 16/30; C07K 16/3007; C07K 16/3092; C07K 16/32; A61K 38/00

(57) ABSTRACT

A heterodimeric fusion protein and the use thereof. The heterodimeric fusion protein includes: a first heavy chain including a first Fc region and an immunomodulator fused to the first Fc region; a light chain and a second heavy chain, the second heavy chain including a second Fc region, and the light chain and the second heavy chain are complexed to form a targeting portion exhibiting the binding specificity to a tumor antigen or an immune checkpoint; and the light chain, the first heavy chain, and the second heavy chain are complexed to form the heterodimeric fusion protein. The heterodimeric fusion protein can be used for the targeted treatment of digestive tract cancer, including colon cancer, and has a high targeting property, and has a tumor inhibition effect significantly superior to that of cetuximab, which has the same target as that of the heterodimeric fusion protein.

### TECHNICAL FIELD

The present invention belongs to the field of biomedicine and specifically relates to a heterodimeric fusion protein and use thereof.

### BACKGROUND

The epidermal growth factor receptor (also known as EGFR, ErbB-1, and HER1) is a cell surface receptor of the ErbB receptor family, a subfamily of four closely related receptor tyrosine kinases, including EGFR (ErbB-1), HER2/c-neu (ErbB-2), Her 3 (ErbB-3), and Her 4 (ErbB-4). Binding of EGFR with ligands (e.g., epidermal growth factor (EGF), transforming growth factor a (TGFα), HB-EGF, dystrophin, β-cytokinin/BTC, epithelial mitogen/EPGN, etc.) induces receptor dimerization and autophosphorylation of a number of tyrosine (Y) residues (Y992, Y1045, Y1068, Y1148, and Y1173) in the C-terminal structural domain of the EGFR. The autophosphorylation triggers downstream activation of a number of signaling cascades including the MAPK, Akt, and JNK pathways, leading to cell migration, adhesion, and cell proliferation.

Mutations, amplification, or misregulation of EGFR or family members are involved in approximately 30% of all epithelial cell cancers. For example, mutations leading to overexpression or overreactivity of EGFR are associated with many cancers, including colon cancer, lung cancer, anal cancer, head and neck cancer, and glioblastoma multiforme. The identification of EGFR as an oncogene has led to the need for the development of anticancer therapeutics targeting EGFR.

Malignant cells that cause disease usually fail to elicit the immune response that leads to rejection. Studies have shown that it is possible to enhance the immunogenicity of tumor cells by introducing immunomodulatory molecules, such as cytokines and co-stimulatory molecules, into the cells; however, eradication of residual cancer cells may require targeting widely dispersed micrometastatic tumor deposits that are not amenable to direct gene transfer. In addition, the expression and stability of introduced immunomodulatory molecules are usually far from satisfactory. Immunomodulators produced by cells of the immune system, such as cytokines, can directly or indirectly activate cells in the adaptive immune response and can play an important role in triggering protective anti-tumor immunity. The innate immune system can be triggered by bacterial products or “danger” signals that lead to the release of pro-inflammatory cytokines such as IFN-α, TNF-α and interleukins.

IL-10 is mainly secreted by activated T cells and antigen-presenting cells, and IL-10 receptor (IL-10R) expression is upregulated in CD8+ T cells during antigen recognition. IL-10 mediates multiple activities by specific cell surface receptor complexes, and the IL-10 receptor contains two distinct chains, IL-10R1 and IL-10R2, both of which belong to the class II cytokine receptor family (CRF2). In bacterial infection and tissue injury, IL-10 reduces inflammatory responses, inhibits inflammatory responses induced by T cells (Th17) and macrophages (IL-12/23), and reduces tumor-associated inflammatory responses. In the tumor microenvironment, IL-10 efficiently activates the proliferation and toxicity of antigen-specific CD8+ T cells.

The anti-tumor mechanism of action of IL-10 is as follows: a. IL-10 can activate the activity and expansion of CD8+ T cells inside the tumor; b. IL-10 can increase the activity and expansion of antigen-specific T lymphocytes inside the tumor; c. IL-10 has a memory function for tumor rejection. Data from animal in vivo tests show that after the disappearance of the tumor given IL-10 drug, the mouse is given again to inoculate the tumor cells, which do not grow in the mouse, and the main reason for this is that IL-10 is able to enhance the survival rate of the antigen-specific CD8+ T cells, which plays the role of a tumor vaccine; d. IL-10 reactivates T cells for the purpose of tumor cell killing by restoring oxidative phosphorylation metabolism in terminally depleted T cells. Clinical trials have also demonstrated that when combined with PDL1 antibody, it increases PDL1-specific CD8+ positive cells in intra-tumor cells, producing a long-lasting anti-tumor effect. However, there are currently no marketed drugs targeting IL-10, which promotes the expansion and viability of CD8+ T cells targeting specific antigens, which are positively correlated with tumor killing by immune cells. Although several studies have demonstrated that immunomodulators can be used to exert antitumor effects in animal models and cancer patients, however, the short half-life and systemic toxicity associated with the use of immunomodulators greatly limits their use. After binding to the receptor, IL-10 initiates the STAT3 and STAT1 pathways, which are signaling pathways in which IL-10 exerts its biological function.

Cancer is a common disease endangering human health, among which the incidence rate of digestive system cancer accounts for more than a quarter of common malignant tumors, and the incidence rate and mortality rate of digestive system cancer are the highest in China. Among them, colon cancer has an insidious onset and often has no obvious clinical symptoms in the early stage, and the disease develops slowly, and most of the patients have already reached the middle or late stage when they have obvious symptoms. At present, no ideal drug has been developed for the treatment of digestive system cancers.

Cetuximab, a monoclonal antibody developed by Merck & Co. targeting the epidermal growth factor receptor, was approved for marketing on Feb. 12, 2004 by the U.S. Food and Drug Administration (FDA). Cetuximab can inhibit the ability of tumor cells to repair damage caused by chemotherapy and radiotherapy, as well as inhibit the formation of neovascularization in tumors, and can be used to treat metastatic colorectal cancer with wild-type RAS and BRAF genes. However, cetuximab has a single target and is not effective enough, so there is still a need to find drugs that are significantly more effective than cetuximab for cancers of the digestive tract.

### SUMMARY

In order to solve the above technical problems, the present invention provides a heterodimeric fusion protein and use thereof. The heterodimeric fusion protein of the present invention can target the treatment of digestive system cancer, especially colon cancer, with good targeting and significantly better tumor inhibition than cetuximab of the same target.

Specifically, the present invention provides a use of a heterodimeric fusion protein in the preparation of a drug for treating or preventing digestive system cancer, wherein the heterodimeric fusion protein comprises: a first heavy chain, wherein the first heavy chain comprises a first Fc region and an immunomodulator fused to the first Fc region; a light chain and a second heavy chain, wherein the second heavy chain comprises a second Fc region, and the light chain and the second heavy chain are complexed to form a targeting portion exhibiting the binding specificity to a tumor antigen or an immune checkpoint; and the light chain, the first heavy chain, and the second heavy chain are complexed to form the heterodimeric fusion protein.

In some embodiments, the tumor antigen or immune checkpoint is one or more of B7H3, B7H4, B7H5, Claudin18.2, BTLA, CD27, CD28, CD153, CD40, CD40L, CD70, CD80, CD86, CD96, CD112, CD134, CD137, CD137L, CD152/CTLA-4, CD155, CD223, CD226, CD252/OX40L, CD258, CD273/PD-L2, CD274/PD-L1, CD278, CD279, CD357, DR3, Galectin-9, ICOSL/B7RP1/B7H2, IDO, TIGIT, TIM-3, TL1A, gp100, TRP-1, TRP-2, BAGE, GAGE-1, GAGE-2, p 15, CEA, Ras, HER-2/neu, MAGE-4, MAGE-5, MAGE-6, RAGE, erbB, p185erbB2, p180erbB-3, c-met, CAM 17.1, NuMa, K-ras, CDK4, Mum-1, p 15, p 16, 43-9F, 5T4, 791Tgp72, B-HCG, BCA225, BTAA, CA 125, MUC16, IL13Ra2, FRa, VEGFR2, LewisY, FAP, EphA2, CEACAM5, CEACAM6, EGFR, CA6, CA9, GPNMB, EGP1, FOLR1, STEAP1, SLC44A4, AGS-16, MUC-1, CFC1B, TPS, CD19, CD20, CD22, CD30, CD72, CD180, CD171, CD123, CD133, CD138, CD37, CD70, CD79a, CD79b, CD56, CD74, CD166, CD71, CLL-1/CLEC12A, ROR1, CD33/IL3Ra, c-Met, PSCA, PSMA, EGFRvIII, BCMA, GD-2, MY-ESO-1; preferably, the tumor antigen is EGFR.

In some embodiments, both the light chain and the second heavy chain comprise a complementary determining region, and the complementary determining region comprises an amino acid sequence having at least 80% identity to the amino acid sequence of the corresponding CDR of the light or heavy chain of the antibody that specifically binds the tumor antigen or the immune checkpoint; preferably, the light chain of the antibody that specifically binds the tumor antigen or the immune checkpoint comprises the amino acid sequence of LCDR1 as shown in SEQ ID NO: 6, the amino acid sequence of LCDR2 as shown in SEQ ID NO: 7, and the amino acid sequence of LCDR3 as shown in SEQ ID NO: 8; preferably, the second heavy chain of the antibody that specifically binds a tumor antigen or an immune checkpoint comprises the amino acid sequence of HCDR1 as shown in SEQ ID NO: 3, the amino acid sequence of HCDR2 as shown in SEQ ID NO: 4, and the amino acid sequence of HCDR3 as shown in SEQ ID NO: 5.

In some embodiments, the light chain comprises a variable region, wherein the amino acid sequence of the variable region of the light chain is as shown in SEQ ID NO: 10, or is an amino acid sequence having at least 80% identity to SEQ ID NO: 10.

In some embodiments, the second heavy chain comprises a variable region, wherein the amino acid sequence of the variable region of the heavy chain is as shown in SEQ ID NO: 9, or is an amino acid sequence having at least 80% identity to SEQ ID NO: 9.

In some embodiments, the amino acid sequence of the light chain is as shown in SEQ ID NO: 13, or is an amino acid sequence having at least 80% identity to SEQ ID NO: 13.

In some embodiments, the amino acid sequence of the second heavy chain is as shown in SEQ ID NO: 12, or is an amino acid sequence having at least 80% identity to SEQ ID NO: 12.

In some embodiments, the first heavy chain comprises one or more immunomodulators of the same or different types. In some embodiments, the immunomodulators are fused to each other and to the first Fc region. In some embodiments, the immunomodulator is IL-10.

In some embodiments, the first Fc region is fused to an immunomodulator by a peptide linker. In some embodiments, the immunomodulator is linked to the N-terminus of the first Fc region by a polypeptide linker. In some embodiments, the polypeptide linker is 5-30 amino acids. In some embodiments, the polypeptide connector is (GGGGS)n, wherein n=1-6.

In some embodiments, the first Fc region and the second Fc region are the same or different. In some embodiments, the first Fc region and the second Fc region is selected from IgG, IgA, IgD, IgE, IgM, or variants thereof. In some embodiments, the first Fc region and the second Fc region is selected from IgG1, IgG2, IgG3, IgG4, or variants thereof. In some embodiments, the first Fc region and the second Fc region comprises one or more amino acid mutations, preferably amino acid substitutions, insertions or deletions. In some embodiments, the first Fc region is knob-Fc and the second Fc region is hole-Fc. In some embodiments, the first Fc region is hole-Fc and the second Fc region is knob-Fc. In some embodiments, the amino acid sequence of the first heavy chain is as shown in SEQ ID NO: 14, or is an amino acid sequence having at least 80% identity to SEQ ID NO: 14.

In some embodiments, the amino acid sequence of the linker monomer in the first heavy chain is as shown in SEQ ID NO: 1. In some embodiments, the amino acid sequence of IL-10 in the first heavy chain is as shown in SEQ ID NO: 2. In some embodiments, the amino acid sequence of the Fc region in the first heavy chain is as shown in SEQ ID NO: 11.

In some embodiments, the digestive system cancer is colon cancer.

The present invention also provides the above-described heterodimeric fusion proteins.

The present invention also provides the above-described heterodimeric fusion proteins which are used for the treatment or prevention of digestive system cancer.

The present invention also provides an isolated polynucleotide which encodes the above-described heterodimeric fusion protein.

The present invention also provides a vector comprising the isolated polynucleotide.

The present invention also provides an isolated host cell comprising the isolated polynucleotide or vector.

The present invention also provides a pharmaceutical composition comprising a pharmaceutically acceptable excipient and the above-described heterodimeric fusion protein.

In some embodiments, the pharmaceutical composition is formulated for oral administration, intravenous administration, intramuscular administration, in situ administration at the tumor site, inhalation, rectal administration, vaginal administration, percutaneous administration, or administration through a subcutaneous reservoir.

The present invention also provides the use of the above-described heterodimeric fusion proteins in the preparation of agents and/or kits for tumor suppression, preferably, the tumor is colon cancer.

The present invention also provides a method of producing a fusion protein comprising (i) culturing a host cell under conditions that achieve expression of the fusion protein, and (ii) harvesting the expressed fusion protein.

The present invention also provides use of the above-described heterodimeric fusion proteins in the preparation of reagents or kits for the detection of tumor antigens or immune checkpoints and IL-10 receptor molecules.

### DETAILED DESCRIPTION OF TERMS

In order to more readily understand the present invention, certain technical and scientific terms are specifically defined below. Unless otherwise expressly defined herein, all other technical and scientific terms used herein have the meanings commonly understood by those of ordinary skill in the art to which the present invention belongs.

The term “heterodimer”, generally refers to a molecule (e.g. a protein molecule) that consists of two different members. The two members of a heterodimer may differ in structure, function, activity and/or composition. For example, the two different members may contain polypeptides that differ in the order, number or type of amino acid residues that form these polypeptides. Each of the two different members of the heterodimer may independently comprise one, two or more units, polypeptide chains or moieties.

The term “fusion protein” refers to a protein comprising one, two or more polypeptides derived from different naturally occurring proteins or engineered proteins artificially combined to form a single protein. It includes, but is not limited to, the following exemplified forms, 1. for a fusion protein comprising a polypeptide chain comprising the same or different polypeptides fused to each other to form a polypeptide chain comprising the different polypeptides; 2. for a fusion protein comprising two or more polypeptide chains, wherein optionally one or more of the polypeptide chains comprises the same or different polypeptides fused to each other to form a polypeptide chain comprising the same or different polypeptides, which polypeptide chains are combined with each other in covalent or non-covalent form to form the protein.

The term “targeting moiety”, generally refers to a molecule, complex or aggregate that specifically, selectively or preferentially binds to a target molecule, cell, particle, tissue or aggregate. For example, the targeting moiety may be an antibody, an antigen-binding antibody fragment, a bispecific antibody or any other antibody-based molecule or compound. Other examples of targeting moieties include, but are not limited to, aptamers, high affinity multimers, receptor binding ligands, nucleic acids, biotin affinity binding pairs, binding peptides or proteins, etc.

The term “tumor antigen”, generally refers to an antigenic substance produced in or by tumor cells that may have the ability to trigger an immune response in the host. For example, a tumor antigen may be a protein, polypeptide, peptide or fragment thereof that forms part of a tumor cell and can induce tumor-specific cytotoxic T lymphocytes. In some embodiments, the term “tumor antigen” may also refer to a biomolecule (e.g., protein, carbohydrate, glycoprotein, etc.) that is uniquely or preferentially or differentially expressed on and/or found to be associated with cancer cells and thus provides a preferential or specific target for cancer. For example, preferential expression may be preferential expression relative to any other cell in the organism, or preferential expression within a specific region of the organism (e.g. within a specific organ or tissue).

The term “immune checkpoint”, generally refers to suppressor and activator molecules in the immune system that regulate the body's anti-tumour immune system through modulation of T-cell activity. For example, suppressor molecules comprise PDL1, B7H3, CTLA4, etc. and activator molecules comprise OX40, 4-1BB, CD40, etc.

The term “Immunomodulator, generally refers to substances that affect the function of the immune system. Immunomodulators may enhance or reduce the immune response. For example, immunomodulators can be active agents in immunotherapy, including, but not limited to, recombinant, synthetic and/or natural preparations of, for example, cytokines, granulocyte colony-stimulating factor (G-CSF), interferon, imiquimod, bacterial cell membrane fragments, chemokines, interleukins, cytosine phosphate-guanosine (CpG) oligodeoxynucleotides and dextran. In some embodiments, wherein the immunomodulator is a cytokine.

The term “polypeptide linker” as used herein, generally refers to a synthetic amino acid sequence that links or couples two peptide sequences (e.g. links two peptide structural domains). A polypeptide linker may link two amino acid sequences through a peptide bond. In some embodiments, the polypeptide linker of the present application links an immunomodulator to the Fc region.

The term “polypeptide linker”, generally refers to a synthetic amino acid sequence that links or couples two peptide sequences (e.g. links two peptide structural domains). A polypeptide linker may link two amino acid sequences through a peptide bond. In some embodiments, the polypeptide linker of the present application links an immunomodulator to the Fc region. The term “antibody” as used herein, generally refers to a protein comprising one or more polypeptides substantially encoded by an immunoglobulin gene or fragment of an immunoglobulin gene. Immunoglobulin genes may include K, 2, a, y, 8, & and u constant region genes and a variety of immunoglobulin variable region genes. As used herein, the light chain may be classified as k or 2. The heavy chain may be classified as y, u, a, 8 or E, which in turn define the immunoglobulin classes: IgG, IgM, IgA, IgD and IgE, respectively. Antibodies as used herein may have structural units comprising tetramers. Each tetramer may comprise two pairs of identical polypeptide chains, each pair having a “light” chain (approximately 25 kD) and a “heavy” chain (approximately 50-70 kD). The N-terminus of each member may define a variable region of approximately 100 to 110 or more amino acids which is primarily responsible for antigen recognition. As used herein, the terms variable light chain (VL) and variable heavy chain (VH) usually refer to these regions of the light and heavy chains, respectively. Antibodies may exist as intact immunoglobulins or as a variety of fully characterised fragments produced by digestion with various polypeptidases or by de novo expression. The term “antibody” as used herein, may also include antibody fragments produced by modification of the whole antibody or by de novo synthesis using recombinant DNA methods, including but not limited to Fab′2, IgG, IgM, IgA, IgE, scFv, dAb, nanobodies, single and double chain antibodies. In some embodiments, the antibodies comprise, but are not limited to, Fab′2, IgG, IgM, IgA, IgE and single chain antibodies, such as single chain Fv (scFv) antibodies, wherein the variable heavy chain and variable light chain are linked together (either directly or via peptide junctions) to form a continuous polypeptide. In some embodiments, the antibodies and fragments of the present application are bispecific. In some embodiments, the bispecific antibody or fragment thereof has binding specificity for at least two different epitopes (e.g., at least one of the at least two different epitopes is a tumor-associated antigen). In some embodiments, the antibodies and fragments may also be heterologous antibodies, e.g. they may be or may comprise two or more linked antibodies or antibody-binding fragments (e.g. Fab), wherein each antibody or fragment has a different specificity.

Kabat is the most commonly used and defines CDRs based on sequence variability; Chothia defines CDRs based on sequence variability based on the position of the structural loop region; the IMGT system defines CDRs based on sequence variability and position within the variable domain structure; AbM is based on Oxford Molecular's AbM antibody modelling software and is a compromise between Kabat and Chothia; Contact defines the CDR based on the analysis of complex crystal structures and is similar to Chothia in several respects. Numbering of amino acid positions (e.g. amino acid residues in the Fc region) and target regions (e.g. CDR) in the present invention, using the Kabat system.

The term ‘identity’ is defined as the percentage of amino acid residues in a candidate sequence that are identical to amino acid residues in a control polypeptide sequence after comparing the sequences and introducing gaps where necessary to obtain the maximum percentage sequence identity. Comparisons for the purpose of determining percentage amino acid sequence identity can be performed in a variety of ways within the skill of the art, for example, using publicly available computer software, such as BLAST software or the FASTA programme package.

The term ‘at least 80% identity’ means that the percentage of amino acid residues in the candidate sequence that are identical to amino acid residues in the control polypeptide sequence is 80% or more, including 80%, 81%, 82%, 83%, 84%, 85%, 86%, 87%, 88%, 89%, 90%, 91%, 92%, 93%, 94%, 95%, 96%, 97%, 98%, 99%, 100%.

The term “host cell”, generally comprises a single cell, cell line or cell culture that will be or has been a recipient of a subject's plasmid or vector comprising a polynucleotide as disclosed herein or expressing a proteinaceous heterodimer of the present application. The host cell may consist of the progeny of a single host cell. The progeny may not necessarily be identical (morphologically or in terms of total genomic DNA complement) to the original parental cells due to natural, accidental or intentional mutations. The host cell may be cells transfected in vitro with a vector as disclosed in this application. The host cells may be bacterial cells (e.g. Escherichia coli (E. coli)), yeast cells or other eukaryotic cells such as COS cells, Chinese hamster ovary (CHO) cells, HeLa cells or myeloma cells.

The term “vector” as used herein, generally refers to a nucleic acid molecule capable of self-replication in a suitable host, which transfers the inserted nucleic acid molecule into and/or between host cells. The term may include vectors primarily for insertion of DNA or RNA into a cell, vectors primarily for replication of DNA or RNA, and expression vectors for transcription and/or translation of DNA or RNA. It also comprises vectors that perform more than one of these functions. An “expression vector” is a polynucleotide that can be transcribed and translated into a polypeptide when introduced into a suitable host cell.

The terms “treatment” refer to methods of achieving beneficial or desired results including, but not limited to, therapeutic benefit and/or preventive benefit. therapeutic benefit generally refers to the elimination or reduction in severity of the underlying condition being treated. In addition, therapeutic benefit is achieved by eliminating, reducing the severity of, or reducing the incidence of one or more physical symptoms associated with the underlying condition such that an improvement is observed in the subject (although the subject may still be afflicted with the underlying condition). For preventive benefits, the combination may be administered to subjects at risk of developing a specific disease, or who report one or more physical symptoms of the disease, even though a diagnosis of the disease may not have been made.

The term “digestive system cancer” refers to tumor-like lesions that occur in the digestive system. Most of them need to be named according to the location of the tumor and its benign or malignant nature, such as esophageal cancer, gastric cancer, colon cancer, rectal cancer, pancreatic cancer, gallbladder cancer, cholangiocarcinoma, small intestinal cancer, colorectal cancer, and so on. In digestive system cancers, epidermal growth factor receptor (EGFR) gene amplification and its protein overexpression are common, and the protein overexpression also has prognostic guidance. In some embodiments, EGFR protein overexpressing digestive system cancer, particularly EGFR protein overexpressing colon cancers, are preferred for the present invention.

### BRIEF DESCRIPTION OF THE DRAWINGS

FIG. 1 shows the fitting curve of the binding activity of the fusion protein to the human EGFR protein.

FIG. 2 shows the fitting curve of the binding activity of the fusion protein to IL-10Ra.

FIG. 3 shows the affinity curve of the fusion protein to the EGFR protein.

FIG. 4 shows the affinity curve of the fusion protein to the IL-10R protein.

FIG. 5 shows the peak plot of double target binding activity detection between the fusion protein and MDA-MB-231 cells.

FIG. 6 shows the peak plot of double target binding activity detection between the fusion protein and IL-10-Reporter-HEK-293 cells.

FIG. 7 shows the results of the fusion protein activated CD8+ T cell secretion perforation protein assay.

FIG. 8 shows the tumor growth curve of MC38 hEGFR transplant tumor model mice after administration of the test substance fusion protein.

### DETAILED DESCRIPTION

### Example 1 Nucleotide Sequences

The target amino acid sequences were converted into nucleotide sequences, and the nucleotide sequences were obtained as SEQ ID NO: 15 (the first heavy chain), SEQ ID NO: 16 (the light chain), and SEQ ID NO: 17 (the second heavy chain), respectively.

### Example 2 Gene Synthesis and Construction of Expression Vectors

The pcDNA3.4-G418 and pcDNA3.1-G418 vectors were used as dedicated vectors for expressing the light and heavy chains of the described multifunctional antibody, respectively. pcDNA3.4-G418 contains the promoter CMVPromoter, the eukaryotic screening marker G418 tag, and the prokaryotic screening tag Ampicilline for the light chain. The pcDNA3.1-G418 vector contains the promoter CMVPromoter, the eukaryotic screening marker G418 tag and the prokaryotic screening tag Ampicilline used for the heavy chain. Nucleotide sequences of antibody-expressing light and heavy chains were obtained by gene synthesis. The vector and the target fragment were double-enzyme-cut with HindIII and XhoI. After recovery, the DNA ligase was used for enzyme ligation, and the E. coli competent cell DH5a was transformed. The positive clones were selected and subjected to plasmid extraction and restriction digestion verification. The recombinant plasmids containing the genes encoding the first heavy chain, the second heavy chain and the light chain of the fusion protein was obtained as pcDNA3.1-G418-1, pcDNA3.1-G418-2, and pcDNA3.4-G418-3, respectively.

### Example 3 Plasmid Extraction

According to the method described in “Molecular Cloning Experiment Guide” (2002, Science Press), the recombinant plasmids containing the above-mentioned target genes were transformed into competent E. coli cells DH5a, and the transformed bacteria were spread and cultured on LB plates containing 100 μg/mL ampicillin. The plasmid clones were selected and cultured in liquid LB medium, shaken at 260 rpm for 14 hours, and the plasmids were extracted from the endotoxin-free plasmid large extraction kit, dissolved in sterile water, and the concentration was determined with a nucleic acid protein quantifier.

### Example 4 Plasmid Transfection, Transient Expression and Antibody Purification

The ExpiCHO was cultured at 37 °C, with 8% CO₂, at 100 rpm to a cell density of 6×10⁶ cells/mL. The constructed vector PCDNA3.1-G418-6-1, PCDNA3.1-G418-6-2, and PCDNA3.4-G418-6-3 were transfected into the above cells using liposomes at a concentration of 1 mg/mL, determined by reference to the ExpiCHO™ Expression System kit. The cells were incubated at 32 °C, with 5% CO₂, and 100 rpm for 7-10 days. The product was replenished once after 18-22 h of transfection and again between day 5 and day 8. The above culture products were centrifuged at 4000 g in a centrifuge and the medium supernatant was filtered through a 0.22 μm filter membrane and collected. The resulting antibody proteins were purified using ProteinA, ionic columns and the eluate was collected.

ProteinA, ionic column purification is performed as follows: cell culture broth is centrifuged at high speed and the supernatant is taken for affinity chromatography using GE's ProteinA chromatography column. The chromatography was performed using an equilibration buffer of 1×PBS (pH 7.4). The cell supernatant was combined and washed with PBS until UV was returned to baseline, then the target protein was eluted using an elution buffer of 0.1 M glycine (pH 3.0) and stored at a neutral pH using Tris. The affinity chromatography product is adjusted to a pH 1-2 pH units below or above the isoelectric point pI and diluted appropriately to control the sample conductance below 5 ms/cm. A NaCl gradient elution at the corresponding pH is carried out using a suitable corresponding pH buffer such as phosphate buffer, acetate buffer, etc., using conventional ion exchange chromatography methods in the art such as anion exchange or cation exchange, and the collection tubes where the target proteins are located are combined and stored according to SDS-PAGE. The collection tubes containing the target proteins were selected for combined storage according to SDS-PAGE and SEC-HPLC.

### Example 5 the Binding Ability of the Fusion Protein to EGFR and IL-10 Receptor Proteins

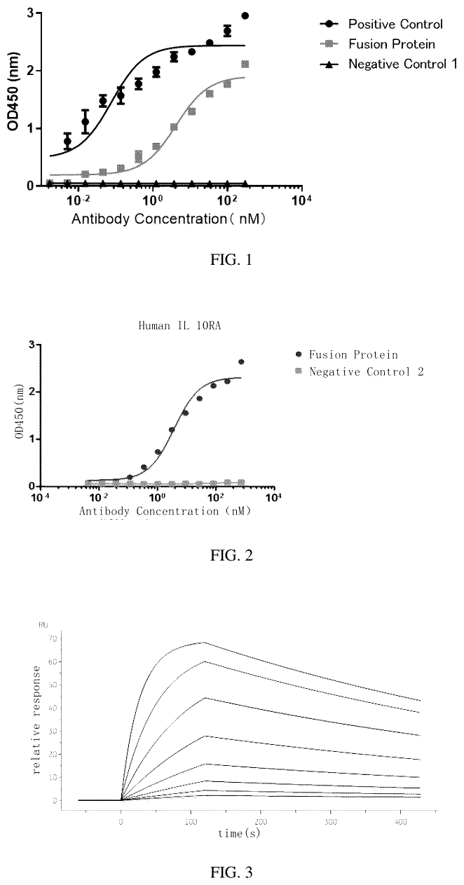

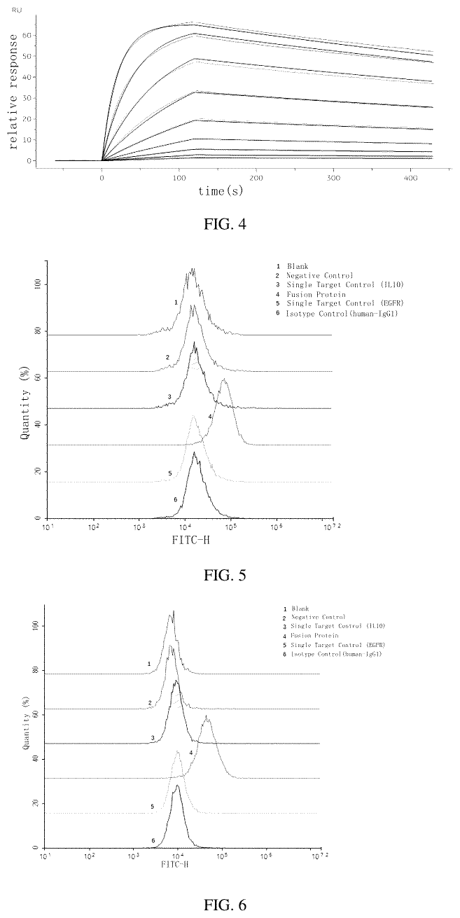

The binding ability of the fusion protein to human EGFR and IL-10R proteins was detected by ELISA, and the affinity of the fusion protein to EGFR and IL-10R proteins, respectively, was detected by SPR technology. The ELISA result shows that the EGFR-binding end of the fusion protein can specifically bind to human EGFR, and the IL-10R-binding end of the fusion protein can specifically bind to human IL-10Ra. The binding activity data are shown in Table 1, and FIGS. 1 and 2. As shown in FIGS. 3 and 4, the affinities of the fusion protein with EGFR and IL-10Ra proteins are 0.51 nM and 3.38 nM, respectively. Among them, the positive control is cetuximab, the amino acid sequences of the heavy and light chains of negative control 1 are shown in SEQ ID NO: 18 and SEQ ID NO: 19, respectively, and the negative control 2 is the commercially available Human IgG1 kappa Isotype Control of Sino, HGK1.

TABLE 1
Binding activity of fusion protein to human
EGFR protein and human IL-10R in vitro

| | Name | | |
|---|---|---|---|
| | | Fusion | Negative |
| Species | Cetuximab | protein | control 2 |
| Human EGFR protein | 0.07751 nM | 4.170 nM | Non-binding |
| Human IL-10R protein | Untested | 3.742 nM | Untested |

### Example 6 Double Target Binding Assay

This assay utilizes the specific binding properties between antigen and antibody and ligand and receptor to analyze the simultaneous binding of the fusion proteins to EGFR and IL-10R by FACS technique using anti-HIS-tagged FITC fluorescent antibody. MDA-MB-231 cells are natural tumor cells that express EGFR antigens, which have been verified to bind to the EGFR end of the fusion protein. IL-10 Reporter-HEK-293 tool cells stably express IL-10R and the STAT3 signaling pathway reporter gene system, which binds to the IL-10 end of the fusion protein. The results of the study show that the IL-10 end of the fusion protein still binds to EGFR in EGFR-positive MDA-MB-231 cells after binding to IL-10RA protein, while the fusion protein still binds to IL-10-Reporter-HEK-293 cells after binding to EGFR protein. The test proved that the fusion protein and its two ends of the action target can bind simultaneously, and after the fusion protein introduced IL-10 into its tumor microenvironment through its anti-EGFR antibody, IL-10 can effectively bind to the action cell and exert its bioactive effect. As shown in FIG. 5 and FIG. 6, the peaks of the fusion protein and MDA-MB-231 cell double-target binding activity and the peaks of the fusion protein and IL-10-Reporter-HEK-293 cell double-target binding activity are shown, respectively.

In FIG. 5, the experiments have been set up with a blank control group (Blank), a negative control group (NC), an experimental group (fusion protein), a single-target control group (cetuximab, Human-IL-10), and an isotype control group (Human-IgG1); the experimental group, single-target control group, isotype control group, and negative control group were first mixed with Human-IL-10 RA Protein (His Tag) protein, then incubated with MDA-MB-231 cells with Anti-6×-His tag antibody (FITC) secondary antibody for 0.5 h, and the secondary antibody binding signal was detected by flow cytometry. Blank controls were added with secondary antibody only and negative controls were protein dilutions. In FIG. 6, the experiments have been set up with a blank control group (blank), a negative control group (NC), an experimental group (fusion protein), a single-target control group (cetuximab, Human-IL-10), and an isotype control group (Human-IgG1); The experimental group, single-target control group, isotype control group and negative control group were first mixed with Human-EGFR Protein, His Tag protein, and then incubated with IL-10-Reporter-HEK-293 cells for 1 h, then Anti-6×-His tag antibody (FITC) secondary antibody was added, and the secondary antibody binding signal was detected by flow cytometry. Blank control group was added with secondary antibody only and negative control group was protein dilution. Human-IgG1 was obtained from commercially available Human IgG1 kappa Isotype Control of Sino, HGK1.

### Example 7 Assay of CD8+ T Cell Activation by Fusion Protein

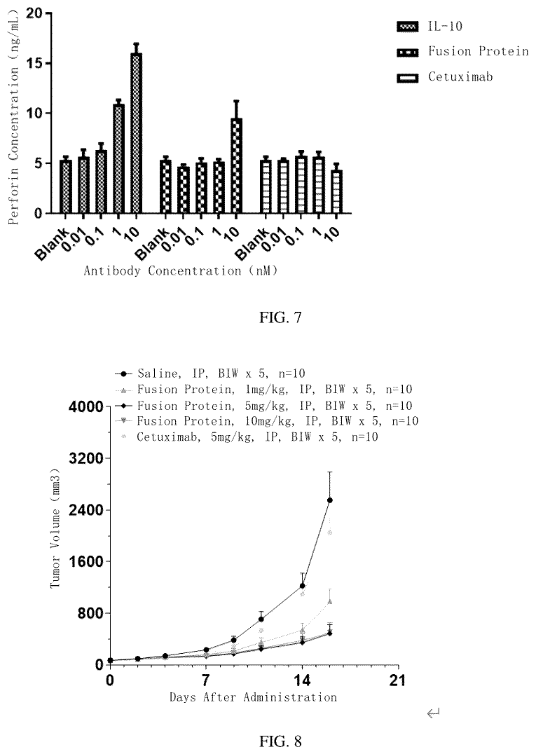

One of the main mechanisms of action of fusion proteins is the activation of CD8+T for tumor cell killing after binding of its IL-10 end to IL-10R in CD8+T. Firstly, the fusion protein was interacted with isolated peripheral blood, and then the stimulated Perforin secretion from the fusion protein to CD8+ T cells was detected using a commercial Perforin cytokine detection kit. As shown in FIG. 7, IL-10 more significantly stimulated CD8+ T cells to secrete perforin in a concentration-dependent manner, the fusion protein antibody could significantly stimulate CD8+ T cells to secrete perforin at high concentrations, while cetuximab could not stimulate CD8+ T cells to secrete perforin, suggesting that the fusion protein stimulated CD8+ T cells to secrete perforin in a role that was dependent on the IL-10 end.

### Example 8 Pharmacodynamic Study of the Fusion Protein In Vivo

One of the mechanisms of action of the fusion protein is that the IL-10 end binds to the IL-10 receptor on the surface of DC cells in the tumor microenvironment and inhibits the apoptosis of CD8+ T cells induced by DC cells, the mouse C57BL/6 was used for the in vivo efficacy of the fusion protein, and the tumor cells were mouse colon cancer cells MC38 overexpressing hEGFR. cetuximab was used as a positive control.

As shown in FIG. 8, compared with the Saline control group, the subject fusion proteins at the doses of 1 mg/kg, 5 mg/kg and 10 mg/kg show significant tumor inhibition, and the TGIs are 63.05%, 83.26% and 82.46%, respectively, whereas the control group of cetuximab shows no significant tumor inhibitory effect with the TGI is 20.15%. The tumor inhibitory effect of the fusion protein 5 mg/kg group is similar to that of the fusion protein 10 mg/kg group, with one of the animals achieving complete tumor remission. Therefore, the tumor-suppressive effect of the fusion protein 5 mg/kg group is significantly better than that of cetuximab, and the main mechanism of action may be the activation of antigen-specific CD8+ T cells in the tumor microenvironment by IL-10.

The protection scope of the present invention is not limited to the above embodiments. Without departing from the spirit and scope of the inventive conception, variations and advantages that can be thought of by those skilled in the art are included in the present invention and are protected by the appended claims.

1. A method of use of a heterodimeric fusion protein in the preparation of a drug for treating or preventing digestive system cancer, wherein the heterodimeric fusion protein comprises: a first heavy chain, wherein the first heavy chain comprises a first Fc region and an immunomodulator fused to the first Fc region; a light chain and a second heavy chain, wherein the second heavy chain comprises a second Fc region, and the light chain and the second heavy chain are complexed to form a targeting portion exhibiting binding specificity to a tumor antigen or an immune checkpoint; and the light chain, the first heavy chain, and the second heavy chain are complexed to form the heterodimeric fusion protein.

2. The method of use of claim 1, wherein the tumor antigen or immune checkpoint is one or more of B7H3, B7H4, B7H5, Claudin18.2, BTLA, CD27, CD28, CD153, CD40, CD40L, CD70, CD80, CD86, CD96, CD112, CD134, CD137, CD137L, CD152/CTLA-4, CD155, CD223, CD226, CD252/OX40L, CD258, CD273/PD-L2, CD274/PD-L1, CD278, CD279, CD357, DR3, Galectin-9, ICOSL/B7RP1/B7H2, IDO, TIGIT, TIM-3, TL1A, gp100, TRP-1, TRP-2, BAGE, GAGE-1, GAGE-2, p 15, CEA, Ras, HER-2/neu, MAGE-4, MAGE-5, MAGE-6, RAGE, erbB, p185erbB2, p180erbB-3, c-met, CAM 17.1, NuMa, K-ras, CDK4, Mum-1, p 15, p 16, 43-9F, 5T4, 791Tgp72, B-HCG, BCA225, BTAA, CA 125, MUC16, IL13Ra2, FRa, VEGFR2, LewisY, FAP, EphA2, CEACAM5, CEACAM6, EGFR, CA6, CA9, GPNMB, EGP1, FOLR1, STEAP1, SLC44A4, AGS-16, MUC-1, CFC1B, TPS, CD19, CD20, CD22, CD30, CD72, CD180, CD171, CD123, CD133, CD138, CD37, CD70, CD79a, CD79b, CD56, CD74, CD166, CD71, CLL-1/CLEC12A, ROR1, CD33/IL3Ra, c-Met, PSCA, PSMA, EGFRvIII, BCMA, GD-2, or MY-ESO-1; preferably, the tumor antigen is EGFR.

3. The method of use of claim 2, wherein both the light chain and the second heavy chain comprise a complementary determining region, and the complementary determining region comprises an amino acid sequence having at least 80% identity to the amino acid sequence of the corresponding CDR of the light or heavy chain of the antibody that specifically binds the tumor antigen or the immune checkpoint; preferably, the light chain of the antibody that specifically binds the tumor antigen or the immune checkpoint comprises the amino acid sequence of LCDR1 as shown in SEQ ID NO: 6, the amino acid sequence of LCDR2 as shown in SEQ ID NO: 7, and the amino acid sequence of LCDR3 as shown in SEQ ID NO: 8; preferably, the second heavy chain of the antibody that specifically binds the tumor antigen or the immune checkpoint comprises the amino acid sequence of HCDR1 as shown in SEQ ID NO: 3, the amino acid sequence of HCDR2 as shown in SEQ ID NO: 4, and the amino acid sequence of HCDR3 as shown in SEQ ID NO: 5.

4. The method of use of claim 1, wherein the light chain comprises a variable region, and the amino acid sequence of the variable region of the light chain is as shown in SEQ ID NO: 10, or is an amino acid sequence having at least 80% identity to SEQ ID NO: 10.

5. The method of use of claim 1, wherein the second heavy chain comprises a variable region, and the amino acid sequence of the variable region of the heavy chain is as shown in SEQ ID NO: 9, or is an amino acid sequence having at least 80% identity to SEQ ID NO: 9.

6. The method of use of claim 1, wherein the amino acid sequence of the light chain is as shown in SEQ ID NO: 13, or is an amino acid sequence having at least 80% identity to SEQ ID NO: 13.

7. The method of use of claim 1, wherein the amino acid sequence of the second heavy chain is as shown in SEQ ID NO: 12, or is an amino acid sequence having at least 80% identity to SEQ ID NO: 12.

8. The method of use of claim 1, wherein the first heavy chain comprises one or more immunomodulators that are fused to each other and to the first Fc region.

9. The method of use of claim 8, wherein the immunomodulator is IL-10.

10. The method of use of claim 8, wherein the first Fc region and the second Fc region are the same or different.

11. The method of use of claim 10, wherein the first Fc region and the second Fc region is selected from IgG, IgA, IgD, IgE, IgM, or variants thereof.

12. The method of use of claim 11, wherein the first Fc region and the second Fc region is selected from IgG1, IgG2, IgG3, IgG4, or variants thereof.

13. The method of use of claim 11, wherein the first Fc region and the second Fc region comprises one or more amino acid mutations, preferably amino acid substitutions, insertions or deletions.

14. The method of use of claim 10, wherein the first Fc region is knob-Fc and the second Fc region is hole-Fc.

15. The method of use of claim 10, wherein the first Fc region is hole-Fc and the second Fc region is knob-Fc.

16. The method of use of claim 1, wherein the amino acid sequence of the first heavy chain is as shown in SEQ ID NO: 14, or is an amino acid sequence having at least 80% identity to SEQ ID NO: 14.

17. The method of use of claim 1, wherein the digestive system cancer is colon cancer.

18. The heterodimeric fusion protein of claim 1.

19. The heterodimeric fusion protein of claim 18, wherein the heterodimeric fusion protein is used for the treatment or prevention of digestive system cancer.

20-24. (canceled)


SEQUENCE LISTING (19 sequences)

SEQ ID NO: 1 - protein, 5 aa, synthetic construct
GGGGS

SEQ ID NO: 2 - protein, 160 aa, synthetic construct
SPGQGTQSENSCTHFPGNLPNMLRDLRDAFSRVKTFFQMKDQLDNLLLKESLLEDFKGYLGCQALSEMIQFYLEEVMPQAENQDPDIKAHVNSLGENLKTLRLRLRRCHRFLPCENKSKAVEQVKNAFNKLQEKGIYKAMSEFDIFINYIEAYMTMKIRN

SEQ ID NO: 3 - protein, 5 aa, synthetic construct
NYGVH

SEQ ID NO: 4 - protein, 16 aa, synthetic construct
VIWSGGNTDYNTPFTS

SEQ ID NO: 5 - protein, 11 aa, synthetic construct
ALTYYDYEFAY

SEQ ID NO: 6 - protein, 11 aa, synthetic construct
RASQSIGTNIH

SEQ ID NO: 7 - protein, 7 aa, synthetic construct
YASESIS

SEQ ID NO: 8 - protein, 9 aa, synthetic construct
QQNNNWPTT

SEQ ID NO: 9 - protein, 119 aa, synthetic construct
QVQLKQSGPGLVQPSQSLSITCTVSGFSLTNYGVHWVRQSPGKGLEWLGVIWSGGNTDYNTPFTSRLSINKDNSKSQVFFKMNSLQSNDTAIYYCARALTYYDYEFAYWGQGTLVTVSA

SEQ ID NO: 10 - protein, 107 aa, synthetic construct
DILLTQSPVILSVSPGERVSFSCRASQSIGTNIHWYQQRTNGSPRLLIKYASESISGIPSRFSGSGSGTDFTLSINSVESEDIADYYCQQNNNWPTTFGAGTKLELK

SEQ ID NO: 11 - protein, 227 aa, synthetic construct
DKTHTCPPCPAPELLGGPSVFLFPPKPKDTLMISRTPEVTCVVVDVSHENPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLASKLTVDKSRWQQGNVFSCSVMHEALHNHYTQKSLSLSPGK

SEQ ID NO: 12 - protein, 449 aa, synthetic construct
QVQLKQSGPGLVQPSQSLSITCTVSGFSLTNYGVHWVRQSPGKGLEWLGVIWSGGNTDYNTPFTSRLSINKDNSKSQVFFKMNSLQSNDTAIYYCARALTYYDYEFAYWGQGTLVTVSAASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHENPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 13 - protein, 214 aa, synthetic construct
DILLTQSPVILSVSPGERVSFSCRASQSIGTNIHWYQQRTNGSPRLLIKYASESISGIPSRFSGSGSGTDFTLSINSVESEDIADYYCQQNNNWPTTFGAGTKLELKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 14 - protein, 567 aa, synthetic construct
SPGQGTQSENSCTHFPGNLPNMLRDLRDAFSRVKTFFQMKDQLDNLLLKESLLEDFKGYLGCQALSEMIQFYLEEVMPQAENQDPDIKAHVNSLGENLKTLRLRLRRCHRFLPCENKSKAVEQVKNAFNKLQEKGIYKAMSEFDIFINYIEAYMTMKIRNGGGGSGGGGSGGGGSSPGQGTQSENSCTHFPGNLPNMLRDLRDAFSRVKTFFQMKDQLDNLLLKESLLEDFKGYLGCQALSEMIQFYLEEVMPQAENQDPDIKAHVNSLGENLKTLRLRLRRCHRFLPCENKSKAVEQVKNAFNKLQEKGIYKAMSEFDIFINYIEAYMTMKIRNGGGGSDKTHTCPPCPAPELLGGPSVFLFPPKPKDTLMISRTPEVTCVVVDVSHENPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLASKLTVDKSRWQQGNVFSCSVMHEALHNHYTQKSLSLSPGK

SEQ ID NO: 15 - DNA, 1758 bp, synthetic construct
  source 1..1758: mol_type = other DNA
atgcactccagcgccctgctgtgctgtctggtgctgctgaccggcgtgagggcttctccaggacagggcacccagtctgagaactcctgcacacatttcccaggcaacctgcccaatatgctgagggacctgcgggatgccttttcccgggtgaagacattctttcagatgaaggaccagctggataatctgctgctgaaggagagcctgctggaggacttcaagggctacctgggctgtcaggctctgtctgagatgatccagttttatctggaggaagtgatgccccaggccgagaaccaggaccctgatatcaaggctcacgtgaacagcctgggcgagaatctgaagaccctgaggctgaggctgaggcggtgccataggttcctgccttgtgagaataagagcaaggccgtggagcaggtgaagaacgcttttaataagctgcaggagaagggcatctacaaggccatgtctgagttcgacatctttatcaactacatcgaggcttatatgacaatgaagatccggaatggcggcggcggctctggaggaggaggatccggaggaggaggcagctctccaggccagggcacccagagcgagaactcttgcacacacttccctggcaacctgccaaatatgctgagagacctgcgcgatgccttttctagagtgaagaccttctttcagatgaaagatcagctggataacctgctgctgaaggagtccctgctggaggatttcaagggttacctgggttgtcaggctctgagcgaaatgattcagttttatctggaggaagtgatgccgcaggctgaaaaccaggaccccgacatcaaggcccacgtgaactctctgggtgaaaatctgaagaccctgagactgcgcctgagacgctgccatcgcttcctgccctgcgaaaataagtctaaagctgtcgaacaggtcaaaaatgcttttaataagctgcaagagaagggcatctataaagctatgtccgaatttgatatctttatcaattatatcgaggcttatatgaccatgaagatcagaaatggcggcggcggctccgataagacccacacatgcccaccttgtccagctccagagctgctgggaggaccttccgtgttcctgtttccacccaagccaaaggacaccctgatgatctcccgcaccccagaggtgacatgcgtggtggtggacgtgagccacgagaaccccgaggtgaagttcaattggtacgtggatggcgtggaggtgcataacgctaagacaaagcctagggaggagcagtacaatagcacctatcgggtggtgtctgtgctgacagtgctgcatcaggattggctgaacggcaaggagtataagtgcaaggtgtctaataaggccctgcctgctccaatcgagaagaccatctccaaggccaagggccagcccagagagcctcaggtgtacacactgcctccaagccgcgaggagatgaccaagaaccaggtgtctctgacatgtctggtgaagggcttctatccctctgatatcgctgtggagtgggagtccaatggccagcctgagaacaattacaagaccacaccccctgtgctggactccgatggcagcttctttctggcctccaagctgaccgtggacaagagcaggtggcagcagggcaacgtgttttcctgtagcgtgatgcatgaggccctgcacaatcattacacacagaagtctctgtccctgagccctggcaagtga

SEQ ID NO: 16 - DNA, 705 bp, synthetic construct
  source 1..705: mol_type = other DNA
atggagtcccagacccaggtgctgatgtccctgctgttctgggtgagcggaacctgcggcgacatcctgctgacacagagcccagtgatcctgagcgtgtctcccggcgagagggtgtcctttagctgtcgggcctctcagtccatcggcaccaatatccactggtaccagcagagaacaaacggcagccctcgcctgctgatcaagtatgctagcgagtctatctccggcatcccatctagattcagcggatctggatccggaaccgacttcaccctgagcatcaattctgtggagtccgaggacatcgccgattactattgccagcagaacaataactggcccaccacattcggcgctggcaccaagctggagctgaagaggacagtggccgctccttccgtgttcatctttcccccttccgacgagcagctgaagagcggcaccgcctctgtggtgtgcctgctgaataacttttacccacgggaggccaaggtgcagtggaaggtggataatgctctgcagagcggcaactctcaggagtccgtgaccgagcaggacagcaaggattctacatattccctgtccagcaccctgacactgtctaaggccgactacgagaagcacaaggtgtatgcttgcgaggtgacccatcagggcctgtcttcccccgtgacaaagtccttcaaccgcggcgagtgttga

SEQ ID NO: 17 - DNA, 1407 bp, synthetic construct
  source 1..1407: mol_type = other DNA
atgggctggagctgcatcatcctgttcctggtggccaccgctacaggcgtgcactctcaggtgcagctgaagcagtccggaccaggactggtgcagccttcccagagcctgtctatcacctgtacagtgtccggcttcagcctgaccaactacggagtgcattgggtgaggcagtccccaggcaagggactggagtggctgggcgtgatctggagcggaggcaacacagactataataccccttttacatctcggctgtccatcaacaaggataattccaagagccaggtgttctttaagatgaacagcctgcagtctaatgacaccgccatctactattgcgccagagctctgacatactatgattacgagttcgcttattggggacagggcaccctggtgacagtgtccgccgctagcaccaagggaccttccgtgttcccactggctccctccagcaagtctacctccggaggaacagccgctctgggatgtctggtgaaggactacttcccagagcccgtgaccgtgtcttggaactccggcgccctgacctccggagtgcacacatttcctgctgtgctgcagtcttccggcctgtacagcctgagctctgtggtgaccgtgccatccagctctctgggcacccagacatatatctgcaacgtgaatcacaagcctagcaatacaaaggtggacaagaaggtggagccaaagtcttgtgataagacccatacatgccccccttgtcctgctccagagctgctgggaggaccatccgtgttcctgtttccacccaagcccaaggacaccctgatgatctctcgcaccccagaggtgacatgcgtggtggtggacgtgtcccacgagaaccccgaggtgaagttcaattggtacgtggatggcgtggaggtgcataacgctaagaccaagccaagggaggagcagtacaattccacctatcgggtggtgagcgtgctgacagtgctgcaccaggattggctgaacggcaaggagtataagtgcaaggtgagcaataaggccctgcccgctcctatcgagaagaccatctctaaggccaagggccagcctagagagccacaggtgtacacactgcctccatctcgcgaggagatgaccaagaaccaggtgtccctgtggtgtctggtgaagggcttttatccctccgacatcgctgtggagtgggagagcaatggccagcctgagaacaattacaagaccacaccccctgtgctggacagcgatggctctttctttctgtatagcaagctgaccgtggataagtctaggtggcagcagggcaacgtgtttagctgttctgtgatgcatgaggctctgcacaatcattacacacagaagtccctgagcctgtctcccggcaagtga

SEQ ID NO: 18 - protein, 451 aa, synthetic construct
QVQLQQPGAELVKPGASVKMSCKASGYTFTSYNMHWVKQTPGRGLEWIGAIYPGNGDTSYNQKFKGKATLTADKSSSTAYMQLSSLTSEDSAVYYCARSTYYGGDWYFNVWGAGTTVTVSAASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKAEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 19 - protein, 213 aa, synthetic construct
QIVLSQSPAILSASPGEKVTMTCRASSSVSYIHWFQQKPGSSPKPWIYATSNLASGVPVRFSGSGSGTSYSLTISRVEAEDAATYYCQQWTSNPPTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC